(19) United States
(12) Patent Application Publication
(10) Pub. No.: US 2007/0087021 A1
(43) Pub. Date: Apr. 19, 2007

(54) METHOD OF PROTECTING AGAINST CHRONIC INFECTIONS

(76) Inventor: Eng-Hong Lee, Guelph (CA)

Correspondence Address: Dennison Associates, Suite 301, 133 Richmond Street West, Toronto, ON, M5H 2L7, CA

(21) Appl. No.: 11/558,894

(22) Filed: Nov. 10, 2006

Related U.S. Application Data
(63) Continuation-in-part of application No. 11/295,511, filed on Dec. 7, 2005, now abandoned.
Application No. 11/295,511 is a continuation-in-part of application No. 09/971,359, filed on Oct. 5, 2001, now abandoned.
Application No. 09/971,359 is a continuation-in-part of application No. 09/062,316, filed on Apr. 20, 1998, now Pat. No. 6,306,385.

(30) Foreign Application Priority Data
Aug. 20, 1997 (CA) 2,213,385

(51) Int. Cl. A61K 39/015; A61K 31/4706; A61K 31/4745

(52) U.S. Cl. 424/272.1; 514/313; 514/305

(57) ABSTRACT

The present invention is directed to a method of protecting an animal against against malaria caused by Plasmodium which undergoes more than one stage in its life cycle or undergoes more than one infectious cycle, the method comprising:
a) administering to the animal a live vaccine containing sufficient Plasmodium sporozites that are low in pathogenicity and sensitive to one or more anti-malarial drugs to develop an immunological response in the animal;
b) maintaining the animal free from anti-malarial drugs effective against Plasmodium for a period of time corresponding to about one life cycle or infectious cycle of the Plasmodium; and
c) thereafter administering to the animal a anti-malarial drugs effective against Plasmodium for a period of time corresponding to at least one life cycle or infectious cycle of the Plasmodium.

### CROSS-REFERENCE TO RELATED APPLICATIONS

This application is a continuation-in-part of U.S. Ser. No. 11,295,511 filed Jul. 12, 2005 which is a continuation-in-part of U.S. Ser. No. 09/971,359, filed Oct. 5, 2001, which is a continuation-in-part of U.S. Ser. No. 09/062,316, filed Apr. 20, 1998, now U.S. Pat. No. 6,306,385 issued Oct. 23, 2001.

### FIELD OF THE INVENTION

The present invention is directed to a method of protecting animals against infections caused by infectious agents which have multiple stages in their life cycle or which may go through multiple infectious cycles, and in particular, a method of protecting against malaria.

### BACKGROUND OF THE INVENTION

There are many diseases which are caused by infectious agents which have multiple stages in their life cycle and/or may go through more than one life cycle during the infection. Such diseases are common amongst human and other animals and are generally caused by infectious agents of various genus and species of protozoa, although in some circumstances, agents of bacterial origin may also cause multiple infectious cycles.

One example of such a disease caused by an infectious agent who has multiple stages in its life cycle is coccidiosis caused by protozoa of the genus Eimeria. Coccidiosis is a very common disease of poultry and there are several species of Eimeria which are known to cause such disease. The symptoms and severity of the disease are dependent upon the species of Eimeria with which the bird is infected with E. tenella, E. acervulina and E. maxima being three of the most prevalent species. Presently, poultry flocks are protected against coccidiosis by either immunization or the use of anti-coccidial chemotherapeutic agents with the most commonly utilized method for controlling coccidial infections being the use of anti-coccidial agents. The study and treatment of coccidiosis is a useful model for other diseases having similar characteristics.

Anti-coccidial agents are commonly ionophores, a class of antibiotics of complex structure although other chemotherapeutic agents are also used. Many such ionophores exhibit anti-coccidial activity, although the relative degree of activity varies from one agent to another. If therapeutic agents such as anti-coccidial ionophores are utilized for control of disease in animals, it is necessary that the agent be continuously administered to the animal for them to be effective. Another problem associated with the use of therapeutic agents is the possibility of resistant strains of the causative organisms developing as a result of exposure to the therapeutic agent. There have, in fact, been reports of resistant strains of Eimeria developing in the field as a result of the use of anti-coccidials.

As noted above, another method of controlling chronic infections is the use of immunization. For example, poultry hatchlings, within the first few days of life, are immunized against various diseases and the type of vaccine used for each disease dictates its method of administration. Attenuated vaccines are usually administered in the hatchery by injection at the time of sorting of the hatchlings from the hatching incubator into holding or transporting trays.

Live vaccines generally comprise live non-attenuated strains of the causative organism, for example coccidia in a suitable carrier for administration, the causative organism being capable of causing a mild form of the disease and selected to be susceptible to suitable antibiotics effective against the causative organism.

Immunization does have some drawbacks, in that there may be antigenic diversity amongst species of the infectious agent as well as amongst strains of a particular species of the infectious agent. Thus, depending upon the antigenic diversity displayed in a field strain of the organism, immunization may not be quite so effective against that particular strain. In addition, organisms may undergo antigenic mutation to the point where the immunological response induced as a result of the immunization will not have sufficient specificity against the antigens present on the field strain to protect the animal against infection by the field strain. Use of live vaccines sometimes results in the induction of a short period of a mild disease state in the animal from which the animal would normally recover, however, in immunosuppressed or immunoincompetent animals the period of the disease state induced by the immunization may be lengthened. In such circumstances the uniformity of the therapy is affected with resultant variation in weight gain and feed conversion of the animals.

For many of these diseases, it is extremely difficult to control the disease through only vaccination or medication. The host relies on infection and immunity to achieve protection from further manifestations of the disease. In order to have immunity, there generally has been an infectious agent present. The difficulty with the vaccines for such organisms causing chronic or multiple life cycle infectious is due to the nature of the organism. Most such organisms have multiple life stages, each with a different antigenic complement. A vaccine which utilizes only an organism at one stage will not provide protection against other stages of the organism. This is particularly true for present dead or subunit vaccines which will not present the full antigenic complement. Thus to be most effective, a live attenuated vaccine is preferred.

There have been attempts to overcome the above difficulty by the use of a combination of immunization and chemotherapy. U.S. Pat. No. 4,935,007, issued Jun. 19, 1990 to Eli Lilly and Company describes a method for control of coccidiosis involving both immunization and ionophore chemotherapy. The method of this patent involves orally administering to the animal at a neonate stage sufficient coccidial organisms to generate an immunological response while maintaining the animal free of any chemotherapeutic anti-coccidial. After the sporozoites have penetrated the host cells, an anti-coccidially effective does of an ionophore is administered substantially continuously throughout the life of the animal.

While the method as described in U.S. Pat. No. 4,935,007 attempts to overcome the difficulties of the two method of controlling coccidiosis, there are drawbacks associated with that method. The ionophore is administered to the animals commencing within 24 hours of immunization and continued throughout the life of the animal. Thus, the use of the method of U.S. Pat. No. 4,935,007 does not result in any significant savings over the traditional use of a chemotherapeutic agent alone. In addition, the commencing of the use of the chemotherapeutic agent within 24 hours of the immunization may not permit the full immunological response to occur, particularly if there are antigens which may not be expressed until later stages of the life cycle.

Another disease in which the causative agent goes through multiple stages during the life cycle, as well as which can induce multiple infectious cycles is malaria, caused by parasites of the genus Plasmodium. It has been estimated that worldwide two billion people are at risk of developing the disease and up to 500 million cases of malaria occur each year. The disease results in the death of 1 to 2 million people annually, mainly children under 5 years of age, but also a significant number of pregnant women. Generally, malaria is controlled by attempts to control the mosquitoes, which are the vector for transmittal of the disease from one human to another and through the use of anti-malarial drug of various quinine derivatives. The risk with the use of such anti-malarial drugs is that the Plasmodium may become resistant to the effects of the drug. Malaria vaccines comprising isolated surface antigens or the pre-erythrocytic stage of asexual blood-stage are presently under development using antigens identified with variant stages of the plasmodium. Such vaccines may confer some degree of immunity but generally suffer the drawbacks of all subunit or killed vaccines. Such vaccines do not present the full antigenic complement of the infectious organism to the host. Rather, they are limited to the antigens specific for the stage of the life cycle or to the antigens expressed during the stage of the life cycle. If the organism can break through the stage being immunized against, the host will have minimal or no immunity against other life cycles.

Live vaccines present the best chance of success for conferring protective immunity against these infectious agents or in the form of immunity now known as infection immunity. However, to be most effective, the live vaccine should mimic the action of the native organism. In many case, this requires that the live vaccine induce a sub-clinical infection in the host. For many diseases which are only minimally lethal and their causative agents, this may not present a major problem if the infection caused by the vaccine should progress beyond the sub-clinical stage. For other disease this may be unacceptable.

There thus remains a need for an improved method of controlling diseases caused by infectious agents which undergo multiple life cycles or multiple infectious cycles in animals, and in particular, a method of providing effective control of malaria.

### SUMMARY OF THE INVENTION

In one aspect, the present invention is directed to a method of protecting an animal against against malaria caused by Plasmodium which undergoes more than one stage in its life cycle or undergoes more than one infectious cycle, the method comprising:
a) administering to the animal a live vaccine containing sufficient Plasmodium sporozites that are low in pathogenicity and sensitive to one or more anti-malarial drugs to develop an immunological response in the animal;
b) maintaining the animal free from anti-malarial drugs effective against Plasmodium for a period of time corresponding to about one life cycle or infectious cycle of the Plasmodium; and
c) thereafter administering to the animal a anti-malarial drugs effective against Plasmodium for a period of time corresponding to at least one life cycle or infectious cycle of the Plasmodium.

### DETAILED DESCRIPTION OF THE PREFERRED EMBODIMENTS

The present invention is directed to a method for protecting an animal against a disease caused by an infectious organism which undergoes more than one stage during its life cycle or which causes more than one infection cycle. The present invention is effective against such organisms, particularly, slowly evolving organic pathogens. Many such diseases are known and in a number of such circumstances, subsequent re-infection after the first life cycle tends to increase the extent of deleterious effects of the infection. While the present invention is suitable for use with any such slowly evolving pathogens causing a chronic infection, it is particularly suitable for controlling disease caused by infectious organisms that undergo multiple stages during the life cycle or the organism in any animal susceptible to disease. Generally, such infectious organisms are parasitic protozoa, which undergo multiple stages during their life cycle of infection of the animal. Examples of such protozoa include those of the class Coccidia, such as Eimeria, or Toxoplasma as well as Plasmodium, the causative agent of Malaria and many other protozoa.

There are many similarities between the Plasmodium species of malaria and the Eimeria species of coccidiosis: they are both obligate intracellular protozoan parasites, they are both Apicomplexa, more importantly both disease conditions are endemic in nature, one is naturally evolved and the other is man-made. In malaria mosquitoes and humans hosts are constantly infecting each other for the benefit of the parasite; similarly commercial poultry are constantly exposed to coccidial oocysts in the litter and create endemic conditions by self infecting or by picking up oocysts from the litter. Age is not a deterrent to re-infection. Commercial birds were reported to come down with coccidiosis at age 2 to 3 years. Similarly in malaria age is no deterrent to re-infection either. Immune response to both diseases is cell mediated, and seemingly there is no immune memory. Both infections appear to follow the sine curve as presented. For example the value a is constant, at about 12-14 weeks, and the severity follows the progression with each subsequent point being less severe then the one immediately in front, unless other disease conditions interfere. Therefore, it is not unexpected that both diseases rely on infection immunity to protect the host. A phenomenon well described for Leishmania major, where a small number of parasites remain in the injected site for the host to remain protected.

When the host finally expels the parasite, the host becomes susceptible again or so called sterile immunity. Therefore there is no surprise that protection against these two diseases early on rely on the constant supply of medication, because of the endemic conditions. The search for alternative method of control later is spurred on by drug-resistance. Here, in the development of vaccines, differences between the controls of these two diseases start to diverge. Development of malaria vaccines up until recently are primarily concerned with vaccine safety and therefore research concentrated mainly on the development of recombinant vaccines. Because of these safety concerns and for not working on live vaccines, only occasional glimpses of success were seen such as when irradiated-sporozoites were used for challenging purposes leading to protective immunity. No such overly concern was placed on the development of vaccines against coccidiosis. The development started with live vaccines and became immediately successful.

There were two developments leading to the successful use of coccidiosis vaccines: repeated low dose exposure to live oocysts and the concept of uniform exposure.

Joyner and Norton (1973) showed that repeated low doses, as little as 1 to 5 oocysts of E. tenella one day apart, evoked protective immunity in vaccinated chicks. However, such approach is commercially too expensive to the poultry industry. To apply this idea of uniform exposure to control coccidiosis to tens of thousands of chickens and turkeys effectively and economically, an idea of putting these low levels of oocysts suspended in edible gums then expose the mixture to day old or young chicks, was conceived (Lee, 1986). This concept of uniform exposure was later evolved into spray in feed, spray in the eyes, gel pucks, water spray and gel spray; all are methods of achieving uniform exposure.

Another reason that is less obvious but equally important is achieving autogenous conditions. When chickens are repeatedly vaccinated with the same vaccines, the condition will lead to autogenous re-infections of oocysts in the litter.

Essentially coccidiosis vaccination is achieved through total population vaccination because any unprotected individual birds become immediately susceptible and will be succumbed to the disease because of the endemic condition constant presence of infective oocysts. There is no immune memory similar to that which works for small pox vaccination. Therefore, the so-called herd vaccination is not sufficient in coccidiosis vaccination as a control because any non-vaccinated member or vaccinated member losing its residual infection immediately becomes susceptible to re-infection. Therefore, use of genetic mutant strains, non-replicative strains or (physiologically deficient strains) which can only create herd vaccination cannot achieve total population vaccination. In addition, protection is short lived and repetitions of vaccination must be frequent to achieve total population vaccination. Endemic conditions which are adverse to the hosts under normal conditions appear here to favor this total population vaccination because of constant availability of oocysts. Another reason why coccidiosis vaccination is successful is because of repeated use of the vaccines and seeding of the vaccine species and strains to make all subsequent vaccinations autogenous in nature. This might have explained why just 4 to 7 species of 3 to 4 more popular vaccine were found to be adequate in controlling coccidiosis in commercial poultry worldwide.

If these two requirements are also essential for malaria vaccination, then any vaccines that cannot achieve these two requirements will have difficulties in maintaining the continuous success. Perhaps, this can explain partly why subunit vaccines, which were sought after for safety reasons, could only obtain partial successes now and then.

This success of coccidiosis vaccination also leads to two or other side benefits for the control of coccidiosis. By using drug-sensitive strains in the vaccines, the field strains, which are largely drug resistant, can be temporarily displaced. Then after using the vaccine for a few flocks, the same anticoccidials can be reused again for a few flocks or until the return of the drug-resistant strains (Mathis and McDougald, 1989, Chapman, 1994). This rotating use of vaccination and medication was further modified (Lee, 2001) to combine vaccination and medication in the same flock. Here, birds were vaccinated as hatchlings and were fed with non-medicated feed, which was then switched to anticoccidial medicated feeds after two weeks, for at least one crop for the purpose of reducing pain and suffering by mitigating possible vaccine reactions. As long as the vaccine strains are sensitive or partly sensitive to a number of anticoccidials, this approach can be used continuously and even to perpetuity. Again, here the vaccine strains can achieve total population vaccination and the barns will provide the endemic condition to make vaccination work.

In summary, the primary success of coccidiosis vaccination appeared to rely on total population vaccination and achieving endemic autogenous environment with the vaccine strains. To achieve total population vaccination, the strains must be able to compete with the field strains or wild-type preferably only mildly pathogenic. Repeated use of vaccine until then lead to autogenous conditions. The combination of vaccination and medication, will reduce pain and suffering from the use of live vaccines. The vaccine strains must therefore be sensitive or partially sensitive to medication in order to use this combination method.

These organisms, because of their multiple state life cycles are difficult to control, especially through immunization. In the past, most of the infections have been controlled either by control of the transmission vector, e.g. mosquito for malaria, or by antibiotics effective against the organism itself. In many cases, the organisms have mutated to be resistant to many of the control agents.

The method of the present invention is practiced by first immunizing the animal by administering a live vaccine containing sufficient organisms to develop an immunological response in the animal. This administration is carried out at a suitable time in the life of the animal, which for the poultry model is preferably shortly after birth or hatching. For other animals, the immunization is carried out at the earliest time in the life of the animal being immunized at which they can develop a competent immune response to the vaccine. The selection of such time will be well known to those of skill in the art. In general, for the poultry model the administration will be carried out within the first 24 hours after birth or hatching. For the purpose of illustration of the invention, coccidiosis in poultry caused by Eimeria will be used, as the disease and organism are well studied and the life cycle of both the disease and host are well studied. However, the invention is also applicable to other similar diseases caused by similar acting organism, i.e. diseases such as malaria caused by organisms which have multiple stages in their life cycle or which can cause multiple infectious cycles.

In a poultry hatchery, at the time of emergence of the hatchlings from their shells in the incubation trays, they are generally examined for defects, immunized with other poultry vaccines by injection and then sorted by sex and placed into holding or transporting trays. Once in the brooding trays, the chicks may be administered live vaccine by means of the watering system, by means of on-feed spray or through the use of a gelled vaccine. Aqueous-based live vaccines are generally particulate in nature and if administered in the watering system, the vaccines should be provided in a composition which will maintain a relatively uniform suspension of the organisms in the vaccine. This is particularly true for coccidiosis vaccines which consist of relatively large oocysts of Eimeria species. Coccidiosis vaccines are usually administered orally for immunizing domestic animals of the avian species from coccidiosis, the vaccine having oocysts of at least one coccidium in relation to which the immunization is desired.

The poultry model of the present invention utilizes a gel form of a coccidiosis vaccine as described in my previous patent application WO 96/25951, published Aug. 29, 1996. This form of a vaccine contains the oocysts of the coccidium diluted and suspended in a gel form which results in maintaining a uniform suspension of the oocysts and consequently, relatively uniform infection of the flock by the oocysts. The gel form of the vaccine is particularly useful with Eimeria species, more particularly with Eimeria tenella or other species such as Eimeria necatrix, Eimeria acervulina, Eimeria maxima, Eimeria brunetti, Eimeria proecox, and Eimeria mitis are also useful. The method of obtaining each of these species is well-known by those skilled in the art. Two or more species of Eimeria can be used simultaneously and in the practice of the present invention, it is generally not necessary to use more than six species together in the same suspension.

The gel form vaccine provides for an easy to handle method of vaccinating poultry hatchlings in the hatchery and is, therefore, suitable for general hatchery workers without any special expertise required. The gel form is produced utilizing an edible polysaccharide gel, preferably a low temperature setting alginate or carrageenan gel, most preferably the kappa carrageenan gel sold as refined carrageenan Bengel MB 910, a water soluble kappa-type carrageenan extracted from the red algae Eucheuma cottonii.

The gel form of the vaccine is prepared by dissolving the gel powder in water at a suitable temperature to effect complete dissolution of the polysaccharide powder. The powder is added to the water at a concentration such that, when mixed with the oocyst suspension and allowed to gel, a relatively soft gel results. The dissolved gel powder and organism suspension are mixed in volumes such that the temperature of the resultant mixture is within the tolerant temperature of the organism. Typically, the dissolved gel powder and oocyst suspension are mixed in a ratio of about 1:1 (v:v) to prepare the gel form of the vaccine.

The gel form vaccine has sufficient levels of the oocysts to provide immunization to the flock. It has been found that each hatchling will consume about 0.5 to 1.5 ml of the gel within a few hours and the concentration of the oocysts in the gel should be such as to provide sufficient organisms in this typical volume to immunize the hatchling. It has been found that between about 50 and 1,000 oocysts per bird provides adequate protection and so it is preferred if the gel form of the vaccine has between about 100 and 500 oocysts per ml of gel, to provide for proper immunization of the flock. Preferably, the gel form of the vaccine contains between about 200 and 300 oocysts per ml of gel, most preferably about 250 oocysts per ml of gel. As the gel form is prepared by mixing the dissolved polysaccharide powder with the oocyst suspension in equal volumes, preferably one volume of a 2 to 5% polysaccharide gel solution is mixed with an equal volume of oocyst suspension containing between about 200 and 1,000 oocysts per ml, more preferably one volume of a 2 to 4 percent polysaccharide solution with an equal volume of oocyst suspension containing between about 400 and 600 oocysts per ml, most preferably a 2.5 percent solution of polysaccharide is mixed with an equal volume of oocyst suspension containing about 500 oocysts per ml.

In the past, treatment of diseases caused by an infectious organism may have involved multiple doses of the vaccine. Although it is possible that the administration of the organisms may be in more than one dose, when practicing the present invention, for most organisms where recycling of the organisms occurs there are no advantages to multiple dosing and a single dose is generally sufficient.

When practicing the method of the present invention with organisms that cause multiple infectious cycles, it is possible to rely on recyling of the organism to provide for multiple exposures of the animals to the organism. This is particularly true in the case of coccidiosis in chickens where the oocysts are shed into he feces at the end of every life cycle. The chicks are thereby exposed to the shed oocysts at the end of the first life cycle, and thus are given a second dose of the organisms. A chemotherapeutic therapy is commenced must after the start of the second life cycle, or just after the animals have been exposed to the organisms which have been shed at the end of the first life cycle. For malaria infections, recycling will occur as a result of the use of mosquitoes as delivery for the vaccine organisms.

The exact number of infective organisms to be administered to the vaccine should be such to be effective to develop an immunological response in the animal. The number should be sufficient to provide a uniformly low level exposure to all animals without causing severe disease. This number would vary, depending upon the nature of the infective organism and the animal to be treated. Such factors may include the species and size of the animal, the relative immunogenicity of the infective organism being administered in the vaccine, the nature of the vaccine, whether the organism is developed from a wild strain or an attenuated strain, and other factors known to those of skill in the art. The suitable number of organisms sufficient to develop the immunological response in the animal may be easily determined by those of ordinary skill in the art through the use of common techniques for vaccine development.

After the infectious organism is administered to the animal, the animal is left to be exposed to the entire antigenic complement of the organism to enable it to develop the full immunological response to the infective organism before the commencement of chemotherapy. Generally, the period of time between administration of the infective organism and the commencement of chemotheraphy would correspond to about one life cycle of infectious cycle of the infective organism. This will enable the animal to be exposed to the full antigenic complement of the infective organism, particularly for those infective organisms which go through various stages in the life cycle. For example, protozoa such as Plasmodium, Toxoplasma or Eimeria will go through a number of stages in the life cycle such as sporulated oocysts, sporozoites and sporocysts for Eimeria. Other protozoa have similar multiple stages during their life cycle. The antigenic complement presented to the animal during each of these stages may be different and it is of advantage to the development of the immunological response that the animal be exposed to the full antigenic complement. In a preferred embodiment of the present invention, the administration of the chemotherapeutic agent is commenced during the early part of the second life cycle or infectious cycle and continued to the end of the third life cycle or infectious cycle. By commencing the chemotherapeutic agent treatment during the second life or infectious cycle, this allows recycling to occur in the animal, such that they are exposed to the organisms shed at the end of the first life or infectious cycle. However, the subsequent stages in the recycling, such as the exposure at the end of the first life or infectious cycle may not be as uniform as the exposure to the controlled dosages of the vaccine. Consequently, the effects of such exposure are more variable.

For Eimeria species, one life cycle takes approximately 5 to 9 days, with major species having life cycles of 5 to 7 days, E. tenella having a life cycle of 6 to 7 days. Thus, in the model of the present invention for controlling coccidial infections in poultry, within about 10 days after administering the vaccine, chemotherapy is begun by administering the suitable chemotherapeutic agent effective against the infective organisms.

The nature of the chemotherapeutic agent will depend upon the disease and the nature of the infectious organisms, as well as on the species of the animal being treated. The selection of the chemotherapeutic agent and the amount to be administered is generally similar to traditional chemotherapy regimes for treatment of such diseases in the particular species of animals.

For example, for the control of coccidiosis in poultry, ionophores such as monensin, narasin, lasalocid, and salinomycin are commonly utilized include laidlomycin, nigericin, grisorixin, dianemycin, lenoremycin, lonomycin, antibiotic X206, albroixinn, septamycin, antibiotic A204, etheromycin, isolasalocid, antibiotic A23187, maduramicion, and many others. In addition, non-ionophore chemotherapeutic agents such as amprolium, diclazupil, clopidol, decoquinate, narasin, nicarbazin, robenidine, halofuginone, and zoalene may also be used. The ionophore anti-coccidial chemotherapeutic agents are preferred. For control of malaria, quinine derivatives such methoquine or chloroquine are commonly utilized.

The chemotherapeutic agent is generally administered to the animal for a period of time corresponding to at least about one life cycle or infectious cycle of the infectious organism. The main function of the administration of the chemotherapeutic agent is to limit the effects to the animal from the live vaccine, particularly, effects evident after the first cycle of the infectious organism. By administering the chemotherapeutic agent at this time, the second and subsequent cycles which generally are not as uniform in exposure in terms of time and dose of exposure are controlled. Such subsequent cycles may also cause cumulative effects which may cause more damage to the animal. This enables mitigation of any possible side effects of vaccination, while permitting the full immunological response of the animal to the vaccination to develop.

Depending upon the nature of the infectious agent, the nature of the disease and the species of animal being treated, the chemotherapeutic agent will be administered to the animal for one or more such life cycles or infectious cycles. Thus, in some circumstances, it may only be necessary to administer the chemotherapeutic agent to the animal for a period of time corresponding to one cycle of the infectious organism. After that time, the continuation of chemotherapy is no longer necessary. In other circumstances, it may be necessary to maintain the animal on the chemotherapeutic agent for a period of time corresponding to more than one life or infectious cycle of the infectious organism, perhaps for a period of time corresponding to two or three such cycles of the infectious organisms.

In particular, using control of coccidiosis in poultry as the model, in most circumstances, it is only necessary to maintain the animals on the chemotherapeutic agent for a period of time corresponding to at least one life cycle of the coccidial organism, preferably for a period of 10 to 14 days. After this time, the birds are immunized and the medication is no longer needed for protection.

In some circumstances, such as for example where the birds are being exposed to a particularly virulent field stain, it is preferable to maintain the animals on the chemotherapeutic agent for a period of time corresponding to about two life cycles of the organism. Thus, for example when the method is used to control coccidiosis in poultry, the birds will be maintained on the chemotherapeutic agent for a period of about 20 to 30 days. After this period of time, the chemotherapy is discontinued and the animals are maintained on a non-medicated feed.

The administration of the chemotherapeutic agent follows the methods of administration commonly employed in the art. Typically, for animals such as cattle, swine, poultry, etc., the chemotherapeutic agent is administered to the animal as an additive to the feed on which the animal is raised, i.e. by using a medicated feed. Alternatively, for some chemotherapeutic agents, they may be administered through the drinking water.

While in most circumstances a single chemotherapeutic agent is utilized in the chemotherapy treatment, there may be circumstances where mixtures of more than one such chemotherapeutic agent may also be employed.

The method of the present invention also has particular utility for replacement of the naturally occurring strains of organism in the field by a particularly selected organism. As the method of the present invention utilizes a live vaccine, for at least the first life cycle of the organism, viable organisms will be shed from the animals into the environment in which the animal is located. Over a period of a number of generations of the animal in the same location, the proportion of such organisms shed as a result of the immunization scheme in the total population of organisms present in the environment will increase until substantially most or all of the organisms in the environment are present as a result of the immunization process. In this way, the nature of the organisms in the environment may be controlled to select for organisms having a particular property, such as increased susceptibility to chemotherapeutic agents.

The method of the present invention is also adaptable to the nature of the organism found in the natural environment in which the animals are located. By utilizing standard techniques, the organisms native to the environment may be isolated and identified. Once the organism is isolated and identified, vaccines specific for the organisms and the particular strains present in the environment may be prepared to maximize the protection of the animal to the organisms found in the environment.

For example, a described in E. Lee, Proceedings of the VIth International Coccidiosis Conference (1993), page 118, if it is found that on a particular poultry farm, E. maxima is the major species of Eimeria present, then the vaccine could be adjusted to have E. maxima as the major component of the vaccine. The particular strain of the E. maxima present on the local farm could also be utilized for the preparation of the specific vaccine. The organisms utilized in the vaccine could also be modified to provide for increased susceptibility to chemotherapeutic agents. In this way, the protection of the animal is maximized as the animal is being immunized against the species and strains of organisms found in the natural environment as well as over a number of generations the wild type strain present in the environment will be gradually replaced by the strain utilized in the vaccine.

In addition, the vaccine may also contain organisms which have been genetically engineered to optimize the protection of the animal to the organisms to which the animal would be exposed in the natural environment. For example, if a particularly virulent strain of the organism is present in the natural environment, the organism for the vaccine could be engineered to utilize a less virulent species or strain of the organism, the less virulent species or strain of the organism in the vaccine also being capable of expressing antigens on its surface which cross-react with, or are specific for the more virulent strain found in the natural environment. This would optimize the protection of the animal to the strain found in the natural environment while minimizing the effects of the vaccine on the animal. One example of a genetically engineered organism is the recombinant coccidian described in my Canadian Patent No. 2,098,773, issued Sep. 21, 1999.

Many of the organisms which cause chronic infection have common characteristics such as minimal cross species protection as well as minimal cross strain protection. Thus immunizing against a particular species or even a strain of a particular species, does not necessarily confer immunity to infections caused by other species of the organism or other strains of the species of the organism.

Another characteristic is that there is minimal apparent memory to prior infections. For example, with malaria, it is quite common to see 3 or 4 bouts of infection in an individual in a year. A similar type of effect is observed in poultry with coccidiosis. This is particularly the case in more vulnerable segments of the population having reduced or compromised immune systems for example, infants and the elderly. The body's defense mechanism against a number of these organisms utilizes a cell mediated immune response rather than the humoral immune response.

The vaccination is accomplished utilizing a drug sensitive strain and the medication is used to control the extent of the infection caused in the body by the organisms used in the vaccine such that the person's immune response can properly respond and confer long term immunity against the organism. In this way, the medication is more effective in treatment. In addition, through strain or species selection, drug resistant strains naturally occurring may be replaced by more sensitive strains and therefore the medication to which the organisms have developed resistance can be continued to be used.

The vaccine species and strains are reproduced by continuous passages without exposure to medication. This method of reproduction has been shown to be effective in coccidiosis. Coccidiosis control is now reaching a point where vaccinated birds can outperform untreated birds in weight gain, egg production, etc.

Through seed control utilizing strain placement by vaccination, there is a reduction in the heterogeneity of the wild field strains. This helps to prevent the wild-type from evolving to develop drug resistance and increase pathogenicity. Conversely, normal drug therapy forces evolution of the wild-type strain to select for a drug resistant strain having increased pathogenicity.

By knowing the strain utilized for the production of the vaccine, the benefit is that reduction of variability of properties of the organisms and an increased knowledge of control of outbreaks is accomplished. The strain is kept pure by passage through medication-free hosts. Knowing the strain used for vaccinations also results in a known medication regime that is most effective to reduce pain and suffering associated with the subclinical infections required to provide proper immunity to the house. This is particularly important for the most at risk or more vulnerable segments of the population for example, infants, elderly, etc.

Preferably the vaccine is produced using antigenic genes to transfer antigenic determinants from more pathogenic strains to less pathogenic strains. This reduces the number of strains or species needed to produce the appropriate vaccine by developing a recombinant strain having full antigenic complement while low pathogenicity and remain relatively prolific. This provides for benefit of increasing the effectiveness of the vaccine. Once such a recombinant strain is developed, and has been used successfully to become the dominant strain of the endemic area, it is also possible then to use a dead vaccine against various stages of this strain to maintain control.

To be most effective, the vaccine is administered replicating the natural route of infection by the organism in the normal cause. In this way, the host develops the proper immunity to the infectious agent.

The method of the present invention is illustrated in the following examples using coccidiosis in poultry as the mode but the invention is not limited thereto. In all of the examples where it is stated that the animals were exposed to oocysts, it should be understood that the vaccine being the oocysts in the gel form was not fed to the bird manually, the birds were simply placed in the same area as the gel form vaccine during the time period and were allowed to consume the vaccine voluntarily.

### EXAMPLE 1

A gel form vaccine was prepared by first adding 200 ml of hot tap water to 5 gm of kappa carrageenan Bengel MB 910 in a container and mixing until the MB 910 had dissolved. To this solution was added 200 ml of a solution containing 500 oocysts per ml of a mixture of Eimeria acervulina, E. maxima and E. tenella and the combined solution was mixed. The solution was then poured into a plastic watering dish and allowed to cool and get at 4° C. This resulted in a gel form of the vaccine containing 1.25 percent MB 910 and 250 oocysts per ml.

### EXAMPLE 2

A paired-barn comparison was conducted between use of a anticoccidial ionophore and the method of the present invention for protecting poultry against coccidiosis. 51,000 birds were divided into two groups. One group was maintained on feed containing 60 mg per kilogram of feed of salinomycin sodium COXISTAC (Pfizer). The second group was administered the vaccine prepared in accordance with Example 1 on Day 1. These birds were maintained on feed with no medication for 10 days. Thereafter, 60 mg per kilogram of salinomycin sodium was utilized in the feed for a further 10 days after which the animals were maintained on non-medicated feed until shipping. A virginiamysin based growth promotant STAFAC (SmithKline Beecham) at a level of 11 mg per kilogram of feed was used as a growth promotant in both groups. The results are for both groups and the comparison between them as shown in Table 1.

TABLE 1

| Particulars | Vaccine/Salinomycin | Salinomycin |
|---|---|---|
| No. of birds placed | 25,500 | 22,950 |
| No. birds shipped | 23,334 | 21,582 |
| Market Age | 42 days | 42 days |
| | 52 days | 52 days |
| Livability (%) | 91.5 | 91.8 |
| Avg. Weight (kg) | 2.81 kg | 2.72 kg |
| Feed Conversion Ratio | 2.16 | 2.16 |
| EEF | 2.29 | 2.23 |

*Flock was reared for 10 days after vaccination with no medication in the
feed. Salinomycin was included in the feed for the next 10 days.
$EEF = European Efficiency Factor = \frac{\% Liveability \times Avg . Market Weight}{FCR \times Market Age}$

From the above, it can be seen that the use of the combined vaccination and the coccidial treatment resulted in a higher efficiency as measured by the European Efficiency Factor which is related to the percent livability, average market weight, feed conversion rate and market age of the birds.

### EXAMPLE 3

The above example was repeated comparing vaccine/monensin treatment with monensin alone and the results from this are shown in Table 2.

TABLE 2

| Particulars | Vaccine/Monesin | Monesin |
|---|---|---|
| No. of birds placed | 22,950 | 22,950 |
| No. birds shipped | 21,352 | 21,582 |
| Sex | Cockerels | Cockerels |
| Market Age | 34 days | 47 days |
| | 51.5 days | |
| Livability (%) | 93.04 | 94.1 |
| Avg. Weight (kg) | avg. - 2.78 kg | 2.44 kg |
| | 45 days - 2.63 kg | |
| | 51.5 days - 2.90 kg | |
| Feed Conversion Ratio | 2.12 | 2.19 |
| EEF | 2.60 | 2.23 |

* Flock was reared for 2 weeks after vaccination with no medication in the feed. Monesin was included in the feed for the next 2 weeks.

Once again, the use of the vaccine and anticoccidial method of the present invention resulted in a better feed conversion ratio and European Efficiency Factor.

### EXAMPLE 4

The number of broiler/roaster crops were raised on either anticoccidial monensin alone, immunization alone or combined immunization/monensin method of the present invention. The results of these are shown in Table 3.

The present invention provides for a method of protecting an animal against a disease caused by an infectious organism which has more than one stage in its life cycle or which undergoes more than one infectious cycle. The method of the present invention is particularly effective for treating poultry flocks against infections in particular, against coccidiosis infection. The method of the present invention helps to overcome some of the drawbacks of each of the individual methods namely, immunization and/or chemotherapeutic therapy by providing a means of controlling the exposure of the animal to the organism and in the preferred embodiment by controlling the nature of the organism which is found in the natural environment. This is accomplished through wild type field strain replacement by the strain utilized in the vaccine used as part of the method of the present invention.

While the present invention has been illustrated in the specific examples for use in protecting poultry, particularly chickens against coccidiosis, it is also useful for other diseases. For example, the method is also usable for protecting poultry against Salmonella, Newcastle Disease, etc.

Similarly, the method of the present invention is useful with other animals in protecting such animals against chronic diseases caused by infectious agents which recycle for more than one life cycle.

The method of the present invention is also useful in the control of malaria by using a live vaccine and thereafter administering an effective anti-malarial agent such as the various quinine derivatives.

The present invention presents a method for protecting against malaria using control of coccidiosis in poultry as a model. This is a valid model as both Plasmodium and Eimeria are Apicomplexan parasites. They are both obligate, intracellular parasites. Both are endemic diseases: coccidiosis by oral and self infection, malaria by cross-infection between human and mosquitoes. Both show features of infection immunity: protective immunity of the host requiring residual infection or persistence of parasites.

Only live vaccines work because residual infection is needed. There is minimal cross-species of cross-strain protection because the parasite only protects against its own kind. This can give rise to Sterile immunity or no residual infection, host again becomes susceptible. Both disease demonstrate minimal immune memory, especially humoral immunity, and thus re-infection can occur at any age if the host is unprotected.

With coccidiosis control as a model, a working malarial vaccine has only two requirements: sporozoites that are drug sensitive and low in pathogenicity. When given in repeated low doses through mosquito bites, these sporozoites will act as a vaccine. Drug sensitivity of these vaccine strains can be maintained into perpetuity in culture and when used in combination with medication, besides reducing pain and suffering, can also provide a long lasting control of malaria.

In practicing the present invention for malaria control, a suitable vaccine containing sporozoites that are low in pathogenicity and sensitive to one or more anti-malarial drugs is developed using standard techniques. The sporozoites are introduced into a suitable vector, such as a controlled mosquito population. The population of animals, such as humans to be protected are exposed to the mosquitos, resulting in a subclinical infection. At a suitable time after exposure to the mosquitoes, the infected population is administered anti-malarial drugs in accordance with the teaching of the present invention. As a result of the method of the present invention, long lasting control of malaria in the treated population can be achieved. The use of the method of the present invention can also result in replacement of the field strains of Plasmodium by the strains used in the vaccine.

Although various preferred embodiments of the present invention have been described herein in detail, it will be appreciated by those skilled in the art, that variations may be made thereto without departing from the spirit of the invention or the scope of the appended claims.

The embodiments of the invention in which an exclusive property or privilege is claimed are defined as follows:

1. A method of protecting an animal against malaria caused by Plasmodium which undergoes more than one stage in its life cycle or undergoes more than one infectious cycle, the method comprising:
a) administering to the animal a live vaccine containing sufficient Plasmodium sporozites that are low in pathogenicity and sensitive to one or more anti-malarial drugs to develop an immunological response in the animal;
b) maintaining the animal free from anti-malarial drugs effective against Plasmodium for a period of time corresponding to about one life cycle or infectious cycle of the Plasmodium; and
c) thereafter administering to the animal a anti-malarial drugs effective against Plasmodium for a period of time corresponding to at least one life cycle or infectious cycle of the Plasmodium.

2. A method as claimed in claim 1 wherein the animal is maintained free from anti-malarial drugs effective against the Plasmodium for a period of time corresponding to about two to five days beyond one life cycle or infectious cycle of the Plasmodium.

3. A method as claimed in claim 2 wherein the anti-malarial drugs effective against Plasmodium is administered to the animal for a period of time corresponding to about 1.5 to 2.5 life cycles or infectious cycle of the Plasmodium.

4. A method as claimed in claim 3 wherein the live vaccine is administered in more than one low dose on subsequent days.
